(19) United States
(12) Patent Application Publication
(10) Pub. No.: US 2015/0257948 A1
(43) Pub. Date: Sep. 17, 2015

(54) ADULT INCONTINENCE GARMENT WITH REMOVEABLE PAD INSERT HAVING LEAKAGE PROTECTION FLAPS

(71) Applicant: Cassandra Steele, Evanston, IL (US)

(72) Inventor: Cassandra Steele, Evanston, IL (US)

(21) Appl. No.: 13/957,928

(22) Filed: Aug. 2, 2013

Related U.S. Application Data
(60) Provisional application No. 61/827,430, filed on May 24, 2013.

(51) Int. Cl. A61F 13/53; A61F 13/72; A61F 13/76; A61F 13/15

(52) U.S. Cl. CPC A61F 13/53; A61F 13/15203; A61F 13/72; A61F 13/76; A61F 2013/15487; A61F 2013/530868

(57) ABSTRACT

An incontinence garment with a replaceable inner pad assembly has a quick-release lower front panel to aid in avoiding accidents. The lower front panel is detachable from the upper front of the garment via cooperating hook and loop or similar fasteners. A fitted, absorbent pad carried within the garment absorbs or contains waste deposited therein during times of incontinence of the wearer, and the pad can be removed and replaced in the garment without removal of the entire garment from the wearer. Impervious flaps on edges of the inner pad help prevent leakage of liquid waste from the pad and garment.

### PRIORITY CLAIM

Applicant claims the benefit of her co-pending provisional application, Ser. No. 61/827,430, filed May 25, 2013.

### FIELD OF THE INVENTION

Adult incontinence garments comprising briefs and shorts fitted with absorbent pads for containing urine and feces against leakage and soiling of outer clothing.

### BACKGROUND OF THE ART

Excretory incontinence is a serious problem for many people, whether it is a continuous problem or an occasional one. Pads such as those available under the Depends® and other brand names are readily available in drug and grocery stores, for fitting into standard underwear. Special incontinence garments, however, have several drawbacks. Often the garments are difficult or time-consuming to open for normal voiding without use of the absorbent pads. Once such a garment is soiled, it must be removed from the person and discarded, and the removal process requires removal also, first, of pants or slacks and shoes, a time-consuming and often cumbersome process. While removable pads are known for infants' diapers, no adult garment is known which has a removable pad fitted within a regular-appearing undergarment.

### SUMMARY OF THE INVENTION

It is an object of this invention to provide an undergarment, particularly for persons suffering occasional or consistent incontinence of the bowel and/or bladder, which can be opened or unfastened quickly and easily and which has a removable inner, absorbent pad to prevent leakage from the undergarment when soiled. The pad is to absorb and contain or hold wet and solid material until the pad can be removed and discarded, while the garment itself can be re-lined with a new pad and then worn without entire removal of the garment from the person.

The present design, as shown in the drawing FIGS. 1-3, avoids the hassles of known incontinence garments by providing quickly-disconnectable hook and loop type attachment strips and a removable incontinence pad fitted closely into the interior of the garment. The garment preferably has absorbent-material flaps on either side, between the person's legs when worn, extending outside the garment and against the user's skin, to better contain leaks. Once the soiled pad is removed it can be replaced easily it with a new, clean one. The outer garment is not discarded and the care-giver need not remove the person's pants and shoes to effect a clean-up.

The invention comprises an undergarment such as cotton briefs, shorts, or panties, which has a lower front panel fitted with hook-and-loop or similar press joining pads or the like, as snaps, to fasten it into place between the user's legs and up the front of the garment. This panel is thus quickly and easily removed when necessary, for voiding into a toilet or for replacement of the absorbent pad.

### BRIEF DESCRIPTION OF THE DRAWINGS

FIG. 1 shows a front plan view of an undergarment, a man's brief, with the lower front portion released from cooperating hook and loop strips which fasten the portion into place for normal wearing.

FIG. 2 shows a plan view of a replaceable assembly comprising a separating layer, partly cut away, an absorbent pad beneath, and an impervious backing material adapted to fit into the garment of FIG. 1.

FIG. 3 shows a plan view of an alternate replaceable absorbent pad, etc., structure, this one having flaps of impervious material at the sides for helping to prevent leakage out of the pad and garment.

### THE PREFERRED EMBODIMENTS

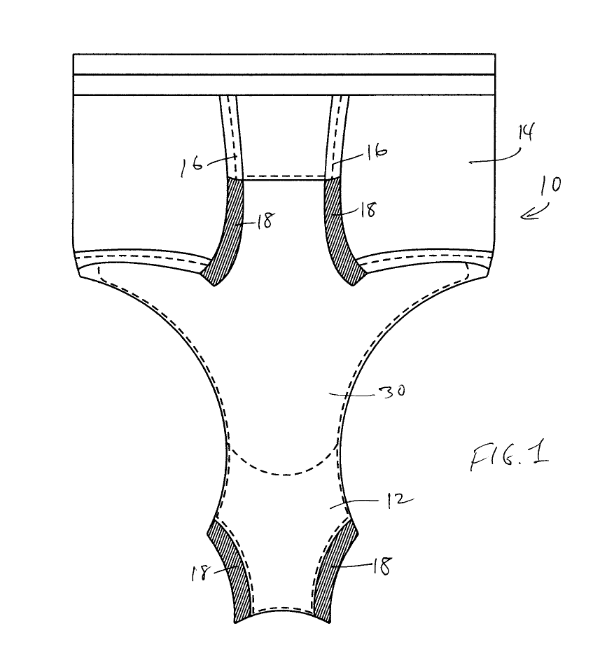

An underwear garment such as a man's brief is modified as shown in FIG. 1. The modified brief 10 has a lower front portion 12 released from an upper portion 14 along regular seam lines 16. Overlapping portions 18 on the upper and lower portions of the brief are fitted with co-operating hook and loop fasteners which stick together when touched and pressed together face to face. Thus, the lower front portion 12 is easily releasable from the upper portion 14 to allow the person to urinate normally, without the delay and complication of removal or other opening of the garment 10 from the person for that purpose when incontinence is not a factor.

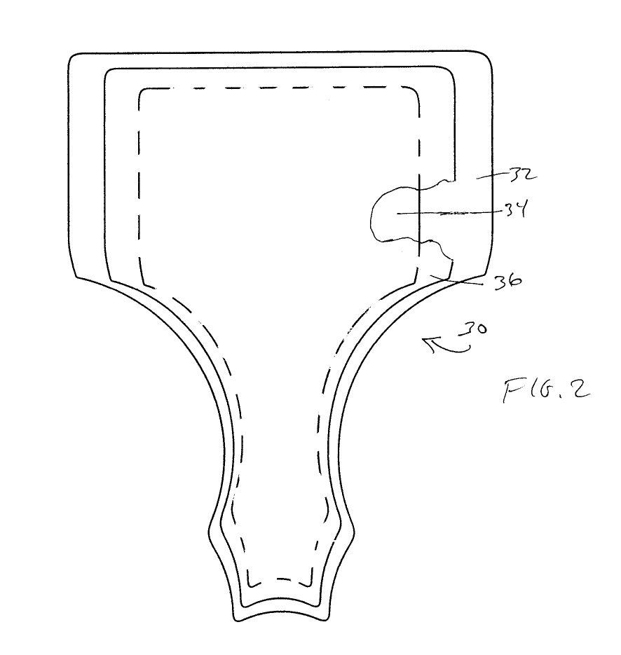

An absorbent pad assembly such as shown at 30 in FIG. 2 is fitted into the brief 10 to receive and absorb liquid waste and to hold solid waste without soiling the undergarment 10 or outer clothing of the person. The pad assembly 30 comprises a rear or outer impervious layer 32, an absorbent center pad layer 34, and a wicking layer 36 adjacent the wearer's skin. All are of conventional material such as that made and sold by TENA®, but are provided in the shape indicated. The pad assembly 20 may be affixed to the garment 10, as by hook and loop fasteners or snaps (not shown) or may simply be positioned appropriately within the garment 10 for use.

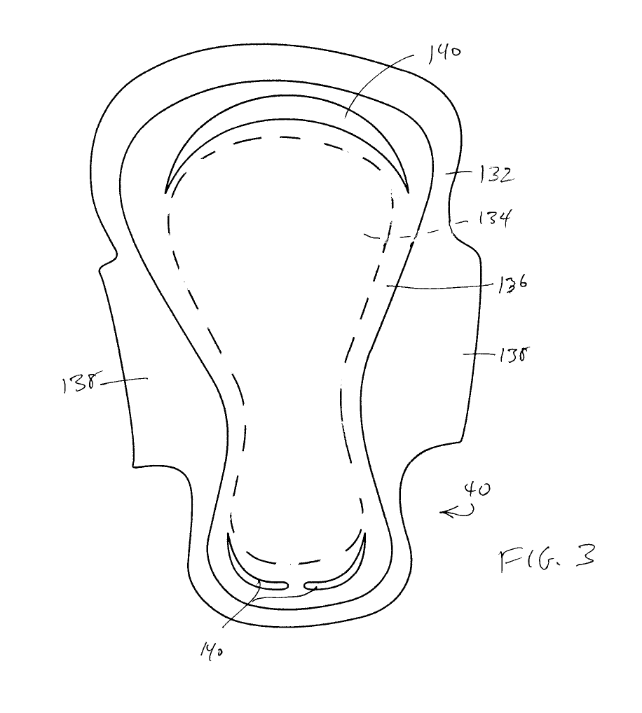

An alternate form of absorbent pad assembly 40 is shown in FIG. 3. The outer layer 132, the absorbent inner pad 134, and the wicking layer 136 are similar in material to those in FIG. 2, but vary in shape, as for a woman's garment. Also, flaps 138 are formed to each side of the outer layer 132 in the area between a user's legs so as to help prevent leakage and soiling of the user's under and outer wear. Stiffening ribs 140 may be provided to help the material of the pad keep its shape.

In use, the pad assembly 40 or 140 is placed into an undergarment such as 10 for absorbing waste during a user's times of incontinence. When the garment is not needed for times of incontinence, the lower portion 12 is simply disconnected quickly and easily from the upper portion 14 by separating the hook and loop fasteners 18-18 from one another along with any supplemental snaps or other fasteners. The garment is then reassembled with the unused pad assembly 30 in its normal position.

When an incontinence event has occurred, the user or a caregiver unfastens the lower portion 12 from the upper portion as above, but takes the pad 30 or 40 from the garment 10 and discards it safely with all contents. A new pad 30 or 40 is placed into the garment and the lower portion 12 is fastened to the upper portion 14 for use until the next voiding event. If a pad with flaps 138 as in FIG. 3 is used, the flaps of the impervious backing material 132 are folded about the edges of the garment 20 to help prevent leakage from the pad 134.

Many variations may be made in the invention as shown and its manner of use, without departing from the principles of the invention as described herein and/or as claimed as the invention. Minor variations will not avoid the use of the invention.

I claim as my invention:

1. An undergarment for a person with bladder and/or fecal incontinence, comprising:
a. an undergarment of cotton or like material and having an upper waistband, enclosing rear, side, and upper front portions below and connected with the waistband, and lower front portions affixed to the rear portion and adapted to pass between the person's legs when worn,
b. at least one hook and loop type attachment strip for affixing an end of the lower front portion opposite the rear portion to the upper front portion of the garment, and
c. an absorbent pad assembly adapted to be fitted within the outer garment, the assembly comprising an impervious outer layer, an absorbent inner pad, and a wicking layer covering the inner pad and affixed to the outer layer about periphery of the pad, adapted to pass liquid waste into the absorbent pad.
whereby the garment may be quickly and easily opened upon a user's need to void, urgently or otherwise, and alternatively the garment and pad and wicking layer retain any waste material deposited into it during a time of incontinence.

2. An undergarment as defined in claim 1, further comprising an absorbent pad fitted removably within the garment for collecting and holding voided matter from the person without substantial leakage of said matter from the pad onto or from the garment.

3. An undergarment as defined in claim 2, wherein the absorbent pad includes flaps extending from either side of the pad in the area passing between the person's legs, each of the flaps passing between the garment and the person's skin, whereby further to avoid leakage from the garment.

4. An incontinence garment with a replaceable absorbent inner pad and lining, the garment having a waistband and upper front and rear portions and an elongated lower front portion selectively disconnectible from the upper front portion and connected to the rear portion, and wherein the replaceable pad is fitted inwardly of the garment and adjacent the person's skin and comprises an impervious outer layer adjacent the garment, an absorbent pad, and a wicking layer adjacent the person's skin for passing liquid waste to the pad.

5. An incontinence garment as defined in claim 4, wherein the replaceable pad has flaps formed on edges of the outer layer extending beyond the garment and wrapping around portions of the garment to protect it from any leakage of waste from the pad and wicking layer.

6. A replacement pad assembly for an incontinence garment, the pad being fitted to the shape of the garment and holding both bladder and excrement waste, the pad assembly having an outer impervious layer for use adjacent the garment, an absorbent pad shaped and fitted to the outer layer, and an inner wicking layer adapted to pass liquid waste to said inner absorbent layer.

7. A replacement pad assembly as defined in claim 6, wherein the pad assembly has flaps extending from the outer impervious layer to the sides of the garment for wrapping about edges of the garment when worn.
